(19) United States
(12) Patent Application Publication
(10) Pub. No.: US 2026/0311806 A1
(43) Pub. Date: Oct. 8, 2026

(54) METHODS FOR IMPROVED DOUBLE HAPLOID PRODUCTION

(71) Applicant: PIONEER HI-BRED INTERNATIONAL, INC., JOHNSTON, IA (US)

(72) Inventors: RICARDO CARRASCO-POBLETE, SEVILLA (ES); ABEBE T. HASSEN, JOHNSTON, IA (US); JEAN-LOUIS LAFFONT, TOULOUSE (FR); OLAF MULLER, JOHNSTON, IA (US); FRANK TECHNOW, CLIVE, IA (US)

(73) Assignee: PIONEER HI-BRED INTERNATIONAL, INC., JOHNSTON, IA (US)

(21) Appl. No.: 19/476,447

(22) PCT Filed: Apr. 19, 2024
(86) PCT No.: PCT/US2024/025385

§ 371 Date: Oct. 17, 2025

Related U.S. Application Data
(60) Provisional application No. 63/497,321, filed on Apr. 20, 2023.

(51) Int. Cl. G16B 20/40; A01H 1/02; G16B 40/20

(52) U.S. Cl. CPC G16B 20/40; A01H 1/02; G16B 40/20

(57) ABSTRACT

The present disclosure provides methods for resource efficient and consistent double haploid production in a high-throughput, industrial setting. In certain aspects, the methods for double haploid production utilize learned prediction models to determine the number of samples in a breeding population needed to produce a desired number of double haploid plants.

### FIELD

The disclosure relates generally to the field of plant breeding, and more specifically to double haploid production.

### BACKGROUND

Double haploid (DH) production is a multi-stage process with low success rates at each stage (e.g., only a proportion of embryos is haploid, only a proportion of haploid embryos can be doubled, only a proportion of doubled haploid embryos survives, and only a proportion of the resulting plants is fertile and can be pollinated). Due to the attrition, the number of samples (seeds, embryos, plants) being processed in each stage must be larger than the target number to produce the number of DHs ultimately desired, which makes DH production a very resource intensive process. Thus far largely constant attrition rates have been assumed for each step when calculating the required number of samples which results in over- or under-sampling in most instances. Over-sampling (applied rate lower than actual rate) leads to overproduction and hence inefficient resource use and waste. Under-sampling (applied rate higher than actual rate) leads to failure of delivering the targeted number of DH plants, which has many downstream consequences, such as a reduced ability to sample and evaluate genetic variation. Accordingly, accurate setting of the attrition rates at the various stages is of utmost importance for resource efficient and consistent DH production in a high-throughput, industrial setting.

### SUMMARY

Provided herein is a method for predicting haploid induction rate or improving haploid production efficiency comprising genotyping a double haploid breeding population comprising a plurality of plants from a parental cross, inputting the genotypic information from the double haploid breeding population into a learned prediction model to generate a predicted haploid induction sampling rate for the double haploid breeding population, calculating based on the predicted haploid induction sampling rate of the double haploid breeding population the number plants in the population that are needed to produce a desired number of haploid embryos, pollinating the calculated number of plants of the double haploid breeding population with an inducer line, and collecting the haploid embryos of the pollinated plants. In certain embodiments, the method further comprises calculating an estimation of the uncertainty of the predicted haploid induction sampling rate and adjusting the predicted haploid induction sampling rate based on the estimation of uncertainty.

Also provided is a method for improved double haploid production efficiency comprising genotyping a candidate double haploid breeding population comprising a plurality of plants from a parental cross, inputting the genotypic information from the double haploid breeding population into a learned prediction model to generate a predicted sampling rate for one or more of haploid induction, haploid doubling, embryo survival, flowering, or harvest, or any combination thereof, calculating based on the predicted sampling rate the number of plants of the candidate double haploid breeding population that are needed to produce a desired number of double haploid plants, pollinating the calculated number of plants of the candidate double haploid breeding population with an inducer line, and producing the desired number of double haploid plants. In certain embodiments, the method further comprises calculating an estimation of the uncertainty of the one or more predicted sampling rates for the double haploid breeding population and adjusting the respective predicted sampling rate based on the estimated uncertainty.

Further provided is a method for improved double haploid production efficiency comprising genotyping a candidate double haploid breeding population comprising a plurality of plants from a parental cross, inputting the genotypic information from the candidate double haploid breeding population into a first learned prediction model to generate a predicted haploid induction sampling rate, inputting the genotypic information from the candidate double haploid breeding population into a second learned prediction model to generate a predicted sampling rate for at least one of, haploid doubling, embryo survival, flowering, or harvest, calculating based on the predicted sampling rates of the double haploid breeding population the number plants in the population that are needed to produce a desired number of double haploid plants, pollinating the calculated number of plants of the candidate double haploid breeding population with an inducer line; and producing the desired number of double haploid plants. In certain embodiments, prior to calculating the method further comprises inputting the genotypic information from the candidate double haploid breeding population into a third learned prediction model to generate a predicted sampling rate for at least one of, embryo survival, flowering, or harvest. In certain embodiments, prior to calculating the method further comprises inputting the genotypic information from the candidate double haploid breeding population into a fourth learned prediction model to generate a predicted sampling rate for at least one of, flowering, or harvest. In certain embodiments, prior to calculating the method further comprises inputting the genotypic information from the candidate double haploid breeding population into a fifth learned prediction model to generate a predicted sampling rate for harvest. In certain embodiments, the method further comprises calculating an estimation of the uncertainty of the predicted haploid induction sampling rate and adjusting the predicted haploid induction sampling rate based on the estimation of uncertainty. In certain embodiments, the method further comprises calculating an estimation of the uncertainty of the predicted sampling for at least one of haploid doubling, embryo survival, flowering, or harvest and adjusting the respective predicted sampling rate based on the estimation of uncertainty.

Provided is a method for predicting embryogenesis induction rate or improving haploid production efficiency comprising genotyping a double haploid breeding population comprising a plurality of plants from a parental cross, inputting the genotypic information from the double haploid breeding population into a learned prediction model to generate a predicted embryogenesis induction sampling rate for the double haploid breeding population, calculating based on the predicted embryogenesis induction sampling rate of the double haploid breeding population the number male gametic cells that are needed to produce a desired number of haploid embryos, inducing androgenesis in the calculated number of male gametic cells of the double haploid breeding population, and collecting the haploid embryos. In certain embodiments, the method further comprises calculating an estimation of uncertainty of the predicted embryogenesis induction sampling rate and adjusting the predicted embryogenesis induction sampling rate adjusted based on the estimation of uncertainty.

Also provided is a method for improved double haploid production efficiency comprising genotyping a candidate double haploid breeding population comprising a plurality of plants from a parental cross, inputting the genotypic information from the double haploid breeding population into a learned prediction model to generate a predicted sampling rate for one or more of embryogenesis induction, haploid doubling, embryo survival, flowering, or harvest, or any combination thereof, calculating based on the predicted sampling rate the number of male gametic cells of the candidate double haploid breeding population that are needed to produce a desired number of double haploid plants, inducing androgenesis in the calculated number of male gametic cells of the candidate double haploid breeding population, and producing the desired number of double haploid plants. In certain embodiments, the further comprises calculating an estimation of uncertainty of the one or more predicted sampling rates for the double haploid breeding population and adjusting the respective predicted sampling rate based on the estimation of uncertainty.

### BRIEF DESCRIPTION OF THE DRAWINGS

The disclosure can be more fully understood from the following detailed description and the accompanying drawings.

FIG. 1 is a block diagram illustrating an exemplary computer system including a server and a computing device according to an embodiment as disclosed herein.

FIGS. 2A and 2B provide histograms of haploid rate data by heterotic group that was used in the development of prediction models for haploid induction sampling rates. FIG. 2A provides data for heterotic group 1. FIG. 2B provides data for heterotic group 2.

FIGS. 3A and 3B provide scatter plots showing the observed versus predicted values for haploid induction along with the corresponding correlation coefficients (r) for the heterotic groups. FIG. 3A provides data for heterotic group 1. FIG. 3B provides data for heterotic group 2.

FIGS. 4A and 4B provide box plots of the observed and predicted data for the haploid induction rate models by heterotic group. FIG. 4A provides data for heterotic group 1. FIG. 4B provides data for heterotic group 2.

### DETAILED DESCRIPTION

A “double haploid”, “doubled haploid” or DH plant or cell refers to a plant or cell that is developed by the doubling of a haploid set of chromosomes. The development of a double haploid plant is a multi-stage process which may include, but is not limited to, haploid induction, haploid doubling, embryo survival, flowering, and harvest. The production of a double haploid plant typically begins with either a haploid induction stage in which a heterozygous plant of interest is crossed with (e.g., pollinated by) an inducer line to produce haploid embryos which are collected or an embryogenesis induction stage in which an anther or male gametic cells (e.g., a tetrad microspore, a single cell microspore, or a pollen grain) are induced to undergo androgenesis to produce haploid embryos which are collected. In a haploid doubling stage, the collected haploid embryos are treated with a doubling agent (e.g., chemical doubling agent) to generate embryos with double the chromosome number. Plants are then generated from the embryos with double the chromosome number or plants are generated by growing haploid plants from the haploid embryos and treating the roots or seedings with a doubling agent in a stage referred to herein as embryo survival. The plants are self-pollinated in a stage referred to herein as flowering and the DH seed, which produces the double haploid plants, from the self-pollinated plants are collected in the harvest stage.

Due to low success rates from each stage, the number of samples being processed in each stage must be larger than the target number to produce the desired number of double haploid plants. For example, only a small percentage of seeds are induced to form haploid embryos and only a small percentage of the haploid embryos can double and form double haploid embryos. Thus, accurate setting of the success rates at the various stages is important for resource efficient and consistent DH production in a high-throughput, industrial setting. Accordingly, the present disclosure provides methods for increasing double haploid production efficiency by using learned prediction models to generate a predicted sampling rate for one or more steps in the double haploid production pathway and calculating, based on the sampling rates, the number of plants or male gametic cells that are needed to produce a desired number of double haploid plants or embryos.

Provided herein is a method for predicting haploid induction rate comprising inputting the genotypic information from a double haploid breeding population into a learned prediction model to generate a predicted haploid induction sampling rate for the double haploid breeding population and calculating based on the predicted haploid induction rate of the double haploid breeding population the number plants in the population that are needed to produce a desired number of haploid embryos. Also provided herein is a method for predicting embryogenesis induction rate comprising inputting the genotypic information from a double haploid breeding population into a learned prediction model to generate a predicted embryogenesis induction sampling rate for the double haploid breeding population and calculating based on the predicted embryogenesis induction rate of the double haploid breeding population the number of male gametic cells (e.g., a tetrad microspore, a single cell microspore, or a pollen grain) that are needed to produce a desired number of haploid embryos.

As used herein, a “double haploid breeding population” refers to a population of plants from which producing a double haploid is desired. In certain embodiments, the double haploid breeding population for use in the methods provided herein is from a parental cross. The type of parental cross used to generate the double haploid breeding population of the methods provided herein can be any type of plant cross used in plant breeding programs, e.g., an F₁ cross, an F₂ cross, an F₃ cross, a backcross, a three-way cross, a four-way cross, or a combination thereof. The parents of the parental cross can be inbred plant lines, hybrid plant lines (e.g., heterozygous lines), double haploid plants, or any combination thereof. In certain embodiments of the methods described herein, the plurality of plants of the double haploid breeding population is from the same parental cross. In certain embodiments of the methods described herein, the plurality of plants of the double haploid breeding population is from two or more (e.g., 2, 3, 4, 5, 10, 20, 40, 50, 100, 150, 200, 500, 1000 or more) parental crosses.

As used herein a “male gametic cell” is any male haploid cell involved in the process of microsporogenesis and microgametogenesis. A male gametic cell may comprise but is not limited to a tetrad microspore, a single cell microspore, or a pollen grain.

As used herein, an “inbred” refers to a line or variety that has been bred for genetic homogeneity. As used herein, a “hybrid” refers to the progeny obtained between the crossing of at least two genetically dissimilar parents, such as, for example, two inbred parents. In certain embodiments of the methods described herein, the plants of the parental cross are elite lines. In certain embodiments, the plants of the parental cross comprise both an elite line and an exotic line. As used herein, an “elite line” “elite variety” or the like is an agronomically superior line that has resulted from many cycles of breeding and selection for superior agronomic performance. As used herein, an “exotic line” “exotic variety” or the like is a strain or germplasm derived from a plant not belonging to an available elite line or strain of germplasm. In the context of a cross between two plants, an exotic germplasm is not closely related by descent to the elite germplasm with which it is crossed. Most commonly, the exotic germplasm is not derived from any known elite line, but rather is selected to introduce novel genetic elements (typically novel alleles) into a breeding program.

As used herein a “learned prediction model” refers to a model that has been trained using historical data to predict likely outcomes. A learned prediction model is usually not a fixed model and is validated or revised regularly to incorporate changes in the underlying data. The type of learned prediction model for use in the methods described herein is not particularly limited and may be any type of learned prediction model that can predict sampling rates. Similarly, the method of training the learned prediction model is not particularly limited. In certain embodiments of the methods described herein, the learned prediction model is trained using a training dataset comprising double haploids with genotypic and phenotypic information. In certain embodiments of the methods described herein, the learned prediction model is trained using a training dataset comprising genotypic information from parental F1 crosses and the corresponding phenotypic information. In certain embodiments, the genotypic information from the parental F1 cross comprises the genotypes of one or each of the parents of the cross. In certain embodiments of the methods described herein, the learned prediction model is trained using a training dataset comprising both double haploids with genotypic and phenotypic information and genotypic information from parental F1 crosses and the corresponding phenotypic information. In certain embodiments, the phenotypic information comprises data regarding number of samples remaining (e.g., known sampling rates) after one or more steps of the DH production pathway.

In certain embodiments, the learned prediction model comprises a machine learning model. Any suitable machine learning model may be used in the in the methods and systems described herein. Types of models include without limitation statistical models, such as probability models, regression models, and those involving deep learning, such as supervised, self-supervised, and unsupervised models, or combinations thereof. In certain embodiments, the machine learning model is a classification model, a regression model, a clustering model, a dimensionality reduction model, a distribution model, for example, a multivariate or univariate Gaussian distribution model, or a deep learning model. In certain embodiments, the deep learning model is part of an ensemble model. In certain embodiments, the deep learning model is an ensemble model comprising two or more models. In some embodiments, the deep learning model is a supervised learning model. The supervised learning model may be a classification or regression model. The machine learning models include support vector machines, neural networks, such as SVM-DA (Support Vector machines) or ANN (Artificial Neural Networks), or deep learning algorithms and the like.

In certain embodiments, the machine learning model for use in the methods described herein is an artificial neural network (ANN). ANNs are configured to synthesize or learn from a plurality of inputs to produce an output. One or more variables in the algorithms can have weights that are applied to each equation and optimized as the neural network is trained. Based on the amount of training information, the deep learning models or networks get better at producing more helpful outputs. In certain embodiments, the ANN includes a plurality of input factors that may be used to train predicted phenotypic information. These factors include, but are not limited to, QTLs, SNPs, haplotypes, yield, and other historical agronomic or breeding phenotypic components.

In certain embodiments, the learned prediction model comprises a linear statistical model. As used herein, a “linear statistical model” is a model that provides a linear relationship between an independent variable (e.g., genotype) and a dependent variable (e.g., survival rate or sampling rate) to predict the outcome of future events. The type of linear statistical model is not particularly limited and may be any linear statistical model known in the art. In certain embodiments, the linear statistical model comprises a logistic generalized linear prediction model. In certain embodiments, the logistic generalized linear prediction model comprises the use of the following formula, referred to herein as Formula 1:

$${s}_{i} = B ( {p}_{i} , {n}_{i} )$$

$${p}_{i} = ℒ ( {β}_{0} + {y}_{i} {β}_{y} + {\sum }_{j = 1}^{M} {z}_{ij} {u}_{j} )$$

Let nᵢ be the number of embryos rescued from the iₜₕ cross and sᵢ the number of haploid embryos among the nᵢ
where pᵢ is the probability of observing the DH production step product (e.g., haploid embryo, double haploid embryo, surviving embryos, flowering plants, harvested DH seeds) for cross i with

$$ℒ ( {β}_{0} + {y}_{i} {β}_{y} + {\sum }_{j = 1}^{M} {z}_{ij} {u}_{j} )$$

being its linear predictor and [symbol] (⋅) denoting the logistic link function;
β₀ is the general intercept;
βy is an effect of an explanatory variable. The use of β_{y} in Formula 1 is optional;
zᵢⱼ denotes the genotype score of marker j for cross i;
uⱼ is the corresponding marker effect; and
B(⋅) denotes the binomial probability function, with nᵢ known.

In Formula 1, β_{y} considers the effect of an explanatory variable or factor. The explanatory variable used in the formula is not particularly limited and may be any observed or manipulated factor that may affect one or more steps in double haploid production. In certain embodiments, the explanatory factor is a continuous variable, such as, for example, year (e.g., regression slope on the year yᵢ in which the iₜₕ cross was produced) or temperature. As an example, in embodiments in which β_{y} accounts for year, a regression slope on the year yᵢ in which the i^{th} cross was produced may be used in Formula 1. In certain embodiments, the explanatory factor is a categorical variable, such as, for example, geographical location. In certain embodiments, a single explanatory factor is used in Formula 1. In certain embodiments, two or more (e.g., 2, 3, 4, 5, 10 or more) explanatory factors are used in Formula 1. When two or more explanatory factors are used the factors may be a continuous variable, a categorical variable, or a combination thereof. In certain embodiments, an explanatory variable is not used in Formula 1 such that β_{y} can be removed from the formula.

A “sampling rate” as used herein, refers to a measure of the number of samples present at the start of any step provided herein compared to the number of samples at the end of the step. The sampling rate may be considered an “attrition rate” in which the expected number of samples that fail to reach the end of the step is measured. The sampling rate or attrition rate can be provided as a probability. In certain embodiments, sampling rate for haploid induction is measured as the number of haploid embryos rescued compared to the total number of embryos rescued. In certain embodiments, sampling rate for embryogenesis induction is measured as the number of haploid embryos rescued compared to the total number male gametic cells induced to undergo androgenesis. In certain embodiments, sampling rate for haploid doubling is measured as the number of embryos with double the chromosome number after treatment with a doubling agent as compared to the total number of haploid embryos treated. As would be understood by a person of ordinary skill in the art the sampling rate for haploid doubling may be dependent on the type of doubling agent used, such that the training data in certain embodiments for predicting haploid doubling sampling rates uses the same doubling agent. In certain embodiments, the sampling rate for embryo survival is measured as the number of plants grown or germinated as compared to the number of embryos with double the chromosome number planted. In certain embodiments, the flowering sampling rate is measured as the number of plants that flower and/or self-pollinate as compared to the total number of plants grown. In certain embodiments, the harvest sampling rate is measured as the number of plants having double haploid seeds that can be grown into a DH plant as compared to the number of plants that flower and/or self-pollinate.

The method for calculating the number of plants in the population that are needed to produce the desired number of double haploid plants is not particularly limited and can be based on the number of sampling rates determined. The following provides an example of a method for calculating the number of plants, if the desired number of DH plants produced from the parental cross is 500 and the sampling rate, provided as a probability, for haploid induction is 0.2, haploid doubling is 0.5, embryo survival is 0.5, flowering is 0.5, and harvest is 0.5 then the number of plants can be determined by dividing 500 (e.g., the desired number of DH plants) by the harvest sampling rate (i.e., 0.5) to determine the number of plants needed at the end of flowering, then taking that value 1000 and dividing by the flowering sampling rate (i.e., 0.5) to determine the number of plants needed at the end of the embryo survival stage, then taking that value 2000 and dividing by the embryo survival sampling rate (i.e., 0.5) to determine the number of haploid embryos with double chromosomes at the end of the haploid doubling stage, then taking that value 4000 and dividing by the haploid doubling sampling rate to determine the number of haploid embryos needed at the end of the haploid induction stage, then taking that value 8000 and dividing by the haploid induction sampling rate (i.e., 0.2) to determine the number of kernels or seeds needed to obtain the desired number of haploid embryos 40,000. The number of plants needed from the parental cross can then be determined by using a fixed number of kernels or seeds per plant or based on the average number of kernels or seeds per plant from the specific parental cross. For example, assuming that the plant is maize and the average number of kernels per plant is 800, then 50 plants would be needed (i.e., 40,000/800). In embodiments in which certain sampling rates are not determined empirically a fixed rate can be used for that step or the step is not included in the calculation of the number of plants.

“Genomic information” or “genotypic profile” as used herein generally refers to a set of information about the entire genome of a given plant or group of plants (genome-wide), or it can encompass a specific subset of the genome of a given plant or group of plants, or any combination thereof in a given plant or group (e.g., population) of plants. In certain embodiments of the methods described herein, the genotypic information of the double haploid breeding population includes information regarding the presence or absence in the genome of a specific set of mutations, single nucleotide polymorphisms (SNPs), insertion of bases, deletion of bases, genotypic markers, other sequence information, or any combination thereof.

The method of genotyping the plants of the double haploid breeding population in the methods described herein is not particularly limited and may be any method known in the art, or described herein, that can determine the genotype of the plant at one or more positions within the genome of the plant. In certain embodiments, one or more plants of the double haploid breeding population are genotyped using molecular biological assays such as, for example, PCR, DNA sequencing, restriction fragment length polymorphism identification or whole genome sequencing. The one or more plants can be genotyped individually or from a pooled DNA sample. In certain embodiments, the parents used to generate the double haploid breeding population are genotyped using molecular biological assays and the genotype of the double haploid breeding population is determined from the parental genotypes. In certain embodiments of the methods described herein, the genotype of the plurality of plants from the double haploid breeding population is simulated. In certain embodiments of the methods described herein, the genotype is simulated using variational autoencoders (VAEs). For example, in certain embodiments of the methods described herein, to predict the genotypes of the double haploid breeding population parental SNPs are imputed using VAEs trained for optimal reconstruction of the population, such as, for example, samples from a breeding program. Non-limiting examples of using VAEs trained for optimal reconstruction include, but are not limited to, those found in U.S. Pat. No. 11,174,522. In certain embodiments of the methods described herein, the VAEs produce intermediate latent representations that could be decoded into imputed SNPs. The genotype of the members of the double haploid breeding population can be simulated from the imputed SNPs, such that in certain embodiments, the genotype of the plants of the double haploid breeding population is imputed from the marker genotypes of the parents of the parental cross. In certain embodiments of the methods described herein, the genotypes of members of the double haploid breeding population can be simulated using a statistical model of recombination. In certain embodiments of the methods described herein, the statistical model of recombination is based on a Poisson process. For example, in certain embodiments to simulate SNPs based on a Poisson process recombination break points in the genetic map may be sampled from an exponential distribution with a rate parameter equal to one, and the sampled distance multiplied by 100 for conversion to centimorgans. In certain embodiments, the crossover interference rate can be sampled from a gamma distribution with shape and scale parameters equal to two. As would be understood by a person of ordinary skill in the art, the exact parameters to simulate the population using a statistical model of recombination based on the Poisson process may be adjusted based on the complexity of the genome of the population. Also contemplated herein are embodiments in which the genotype, or allele frequency, of the product from one or more steps of the DH production pathway is determined, such that the genotype of the double haploid breeding population may be adjusted prior to inputting into the learned prediction model for the respective step or steps. Methods to genotype the products (e.g., haploid embryo, double haploid embryo, surviving plant, and/or flowering plant) from the various steps is not particularly limited and may be any method described herein or known in the art, such as, for example, molecular biological assays. Similarly, the genotypes may be determined individually, or in a pooled sample.

In certain embodiments of the methods described herein, when two or more sampling rates are calculated the genotypic information from the double haploid population is the same for determining the respective sampling rates. In certain embodiments of the methods described herein, when two or more sampling rates are calculated the genotypic information from the double haploid population is adjusted prior to determining the sampling rate for one or more steps of the DH production pathway. In certain embodiments, the genotypic information is adjusted once and the adjusted information is used for the subsequent steps, for example, the genotypic information for the induced haploids is determined and that genotypic information is used to determine the sampling rate for the remaining determined rates. In certain embodiments, the genotypic information is adjusted two or more times. In certain embodiments, the genotypic information is adjusted prior to each step, such that an adjusted genotype of the double haploid breeding population is used to determine each sampling rate.

The plant of the methods described herein is not particularly limited and may be any plant for which production of a double haploid is desired. Examples of plant species of interest include, but are not limited to, maize (Zea mays), wheat (Triticum aestivum), soybean (Glycine max), barley (Hordeum vulgare), sunflower (Helianthus annuus), sorghum (Sorghum bicolor, Sorghum vulgare), potato (Solanum tuberosum), pea (Lathyrus spp), rice (Oryza sativa), Brassica sp. (e.g., B. napus, B. rapa, B. juncea), and cotton (Gossypium barbadense, Gossypium hirsutum). In certain embodiments, maize, soybean, sorghum, sunflower, rice, wheat, and canola, are optimal, and in yet other embodiments maize plants are optimal.

In certain embodiments, the method further comprises pollinating the calculated number of plants of the double haploid breeding population with an inducer line and collecting the haploid embryos of the pollinated plants. An “inducer line” as used herein refers to a plant line genetically capable of causing (inducing) the formation of haploid embryos after gametic fertilization. The inducer line of the methods described herein is not particularly limited and may be any inducer line known in the art. In certain embodiments, the method further comprises generating double haploids from the haploid embryos.

In certain embodiments, the method further comprises inducing androgenesis in the calculated number of male gametic cells (e.g., a tetrad microspore, a single cell microspore, or a pollen grain) and collecting the generated haploid embryos. As used herein, “androgenesis” refers to the process in which male gametic cells give rise to embryos without contribution from female cells. Methods for inducing androgenesis for use in the methods described herein may be any method known in the art such as, for example, those described in U.S. Pat. No. 11,477,786, US Patent Application Publication No. 20220240267, and US Patent Application Publication No. 20220154203. In certain embodiments, the method further comprises generating double haploids from the haploid embryos.

In certain embodiments of the methods described herein, the method further comprises calculating an estimation of the uncertainty, also referred to herein as risk, of one or more of the predicted sampling rates (e.g., haploid induction rate, embryogenesis induction rate, haploid doubling, embryo survival, flowering, or harvest) for the double haploid breeding population. The estimation of uncertainty for the one or more predicted sampling rates may be calculated using any method known in the art. In certain embodiments, the estimation of uncertainty for one or more of the predicted sampling rates is a pre-determined fixed value, such that is certain embodiments a calculated estimation of uncertainty is used for less than all of the predicted sampling rates (e.g., only for haploid induction). In certain embodiments, the estimation of uncertainty is estimated for the one or more sampling rates, either individually, jointly (e.g., estimation of uncertainty is determined for two or more sampling rates in a single calculation), or a combination thereof, using the following formula:
let:
X be a random variable (haploid rate) distributed according to a distribution with cdf F.
x_{α} be haploid rate value such that we are (1−α)×100% confident the haploid rate is greater than this value:

$${x}_{α} = {F}_{α}^{- 1} , i . e . P ( X \leq {x}_{α} ) = α .$$

Now assume that X~-N(μ,σ²).

Then

$$P ( X \leq {x}_{α} ) = P ( \frac{X - μ}{σ} \leq \frac{{x}_{α} - μ}{σ} ) = P ( Z \leq \frac{{x}_{α} - μ}{σ} ) = α ,$$

where Z~N(0,1), and
x_{α}=Φ⁻¹(α)σ+μ, where Φ⁻¹(x) is the cdf of the N(0,1).

In certain embodiments, the one or more predicted sampling rates are adjusted based on the estimated uncertainty. In certain embodiments, the one or more predicted sampling rates are adjusted by calculating the lower limit for the predicted sampling rate at different probability levels. The probability level selected for the lower limit is not particularly limited and can be selected by a person or ordinary skill in the art based on their desired risk allowance. In certain embodiments, the lower probability levels are selected from the group comprising 0.95, 0.90, 0.85, 0.80, and 0.75.

The desired number of double haploid plants or embryos for the methods described herein can be any number of DH plants. The determination of the desired number of DH plants may be based on the plant species, breeding program strategies, and/or downstream processes. In certain embodiments, the desired number of plants is at least 5, 10, 20, 40, 80, 100, 500, 1000, or 2500 plants and no more than 10,000, 5000, 2500, 1000, 500, 100, or 50 plants.

Also provided herein is a method for improved double haploid production efficiency comprising inputting the genotypic information from the double haploid breeding population into a learned prediction model to generate a predicted sampling rate for haploid induction, embryogenesis induction, haploid doubling, embryo survival, flowering, or harvest, or any combination thereof and calculating based on the predicted sampling rates the number of plants or male gametic cells, of the candidate double haploid breeding population that are needed to produce a desired number of double haploid plants. In certain embodiments, the method further comprises pollinating the calculated number of plants of the candidate double haploid breeding population with an inducer line and producing the desired number of double haploid plants. In certain embodiments, the method further comprises further comprises inducing androgenesis in the calculated number of male gametic cells (e.g., a tetrad microspore, a single cell microspore, or a pollen grain) and producing the desired number of double haploid plants.

In certain embodiments, the method comprises generating a predicted sampling rate for at least two of haploid induction, haploid doubling, embryo survival, flowering, and harvest. In certain embodiments, the method comprises generating a predicted sampling rate for each of haploid induction, haploid doubling, embryo survival, flowering, and harvest. In certain embodiments, the method comprises generating a predicted sampling rate for each of haploid induction, embryo survival, flowering, and harvest. In certain embodiments, the method comprises generating a predicted sampling rate for each of embryogenesis induction, haploid doubling, embryo survival, flowering, and harvest. In certain embodiments, the method comprises generating a predicted sampling rate for each of embryogenesis induction, embryo survival, flowering, and harvest. In certain embodiments, when sampling rates are calculated for two or more steps in the double haploid production pathway the sampling rates are determined using separate learned prediction models, combined learned prediction models (e.g., predicted sampling rate for two or more steps using a single learned prediction model), or a combination thereof. In certain embodiments, the predicted sampling rates for haploid induction, haploid doubling, embryo survival, flowering, and harvest are determined in a single learned prediction model. In certain embodiments, the predicted sampling rates for embryogenesis induction, haploid doubling, embryo survival, flowering, and harvest are determined in a single learned prediction model. The learned prediction model may be any learned prediction model described herein or known in the art. In certain embodiments, the learned prediction model comprises a machine learning model, a linear statistical model, such as, for example, a logistic generalized linear prediction model, or a combination thereof. In certain embodiments the logistic generalized linear prediction model comprises the use of Formula 1. In certain embodiments in which a combined learned prediction model is used or the sampling rates are determined in a single model the sampling rates are determined using a Markov process model.

In certain embodiments, the method further comprises calculating an estimation of the uncertainty of the predicted sampling rate for one or more of haploid induction, embryogenesis induction, haploid doubling, embryo survival, flowering, or harvest, or any combination thereof. In certain embodiments, the predicted sampling rate of the one or more steps (e.g., haploid induction, embryogenesis induction, haploid doubling, embryo survival, flowering, or harvest) is adjusted based on the estimated uncertainty. The method for calculating the uncertainty of the respective predicted sampling rate may be any method described herein or known in the art.

Further provided herein is a method for improved double haploid production efficiency comprising inputting genotypic information from a candidate double haploid breeding population into a first learned prediction model to generate a predicted haploid induction sampling rate or embryogenesis induction sampling rate, inputting the genotypic information from the candidate double haploid breeding population into a second learned prediction model to generate a predicted sampling rate for at least one of, haploid doubling, embryo survival, flowering, or harvest, and calculating based on the predicted sampling rates of the double haploid breeding population the number plants in the population or male gametic cells that are needed to produce a desired number of double haploids.

In certain embodiments, prior to the calculating step, the method further comprises inputting the genotypic information from the candidate double haploid breeding population into a third learned prediction model to generate a predicted sampling rate for at least one of, haploid doubling, embryo survival, flowering, or harvest. In certain embodiments, the third learned prediction model generates a predicted sampling rate for a different step in the double haploid production pathway than the second learned prediction model. In certain embodiments, prior to the calculating step, the method further comprises inputting the genotypic information from the candidate double haploid breeding population into a fourth learned prediction model to generate a predicted sampling rate for at least one of, haploid doubling, embryo survival, flowering, or harvest. In certain embodiments, the fourth learned prediction model generates a predicted sampling rate for a different step in the double haploid production pathway than the second and/or third learned prediction models. In certain embodiments, prior to the calculating step, the method further comprises inputting the genotypic information from the candidate double haploid breeding population into a fifth learned prediction model to generate a predicted sampling rate for at least one of, haploid doubling, embryo survival, flowering, or harvest. In certain embodiments, the fifth learned prediction model generates a predicted sampling rate for a different step in the double haploid production pathway than the second, third and/or fourth learned prediction models.

In certain embodiments, the first, second, third, fourth and/or fifth learned prediction models are independent learned prediction models, such that a sampling rate is generated independently for each step or steps in the double haploid production pathway. In certain embodiments, the first, second, third, fourth and fifth learned prediction models comprise a combination of independent learned prediction models and combined learned prediction models, such that a sampling rate is generated independently for at least one step in the double haploid production pathway and a sampling rate for two or more additional steps are determined in a single learned prediction model. For example, in certain embodiments, the haploid induction step is determined independently in a learned prediction model and two or more of haploid doubling, embryo survival, flowering, or harvest are determined in a single learned prediction model. In certain embodiments, the sampling rates are determined jointly for each step in the double haploid production pathway, such that the first, second, third, fourth and fifth learned prediction models are the same model. In embodiments in which the sampling rates of two or more steps are calculated in a single model, the model may provide a single sampling rate for the combined steps or, alternatively, the model may provide a sampling rate for the first step and a sampling rate for the second step.

The type of learned prediction model for use in the methods is not particularly limited and may be any method described herein or known in the art. In certain embodiments, the first, second, third, fourth and/or fifth learned prediction model comprise a machine learning model, a linear statistical model, a logistic generalized linear prediction model, a Markov process model, or any combination thereof. In certain embodiments, at least one of the first, second, third, fourth or fifth learned prediction models comprises a linear statistical model. In certain embodiments, the linear statistical model comprises a logistic generalized linear prediction model. In certain embodiments, the logistic generalized linear prediction model uses Formula 1, described above.

In certain embodiments, the method further comprises calculating an estimation of the uncertainty of the predicted haploid induction or embryogenesis induction sampling rate and/or the predicted sampling rate for one or more of haploid doubling, embryo survival, flowering, or harvest, or any combination thereof. In certain embodiments, estimation of uncertainty is calculated for haploid induction. In certain embodiments, the estimation of uncertainty is calculated for haploid induction or embryogenesis induction and at least one of haploid doubling, embryo survival, flowering, or harvest. In certain embodiments, the estimation of uncertainty is calculated for each of haploid induction, haploid doubling, embryo survival, flowering, or harvest. In certain embodiments, the estimation of uncertainty is calculated for each of embryogenesis induction, haploid doubling, embryo survival, flowering, or harvest. In certain embodiments, the predicted sampling rate of the one or more steps (e.g., haploid induction, embryogenesis induction, haploid doubling, embryo survival, flowering, or harvest) is adjusted based on the estimated uncertainty. The method for calculating the uncertainty of the respective predicted sampling rate may be any method described herein or known in the art. The method for adjusting the sampling rates based on the uncertainty may be any method described herein, including using the calculated uncertainty rates, the fixed uncertainty rates, or a combination thereof.

The method for generating the genotypic information for the double haploid breeding population may be generated using any method known in the art or described herein. In certain embodiments, the genotypic information from the double haploid breeding population is produced by genotyping a double haploid breeding population comprising a plurality of plants from a parental cross, as described herein.

In certain embodiments, the double haploid plants generated by the methods described herein are used in a breeding program. In certain embodiments, one or more of the double haploid plants generated by the methods described herein are self-pollinated. In certain embodiments, one or more of the double haploid plants generated by the methods described herein are crossed with a second plant line to produce progeny plants, such as, for example, F1 hybrid plants.

Also disclosed are computer readable mediums having stored thereon instructions that, when executed by a processor (or computing device), cause the processor to perform the steps of the methods described herein to provide recommendations on the number of plants or male gametic cells that are needed to produce a desired number of haploid embryos or double haploid plants.

Further disclosed herein are systems (e.g., computer systems) for use in double haploid production that include (a) one or more servers, each of the one or more server storing plant data, and (b) a computing device communicatively coupled to the one or more servers, the computing device including: (1) a memory, and (2) one or more processors configured to perform operations to: (a) obtain data from a plurality of candidate plant genotypes (e.g., plurality of plants from a parental cross), (b) generate a sampling rate for one or more of haploid induction, embryogenesis induction, haploid doubling, embryo survival, flowering, or harvest, or any combination thereof using at least one learned prediction model, and (c) calculating the number of plants or male gametic cells that are needed to produce a desired number of haploid embryos or double haploid plants based upon the one or more sampling rates. In certain embodiments, the one or more processors are further configured to perform the operation of calculating an estimation of the uncertainty of the one or more predicted sampling rates and adjusting the respective sampling rate based upon the estimation of uncertainty.

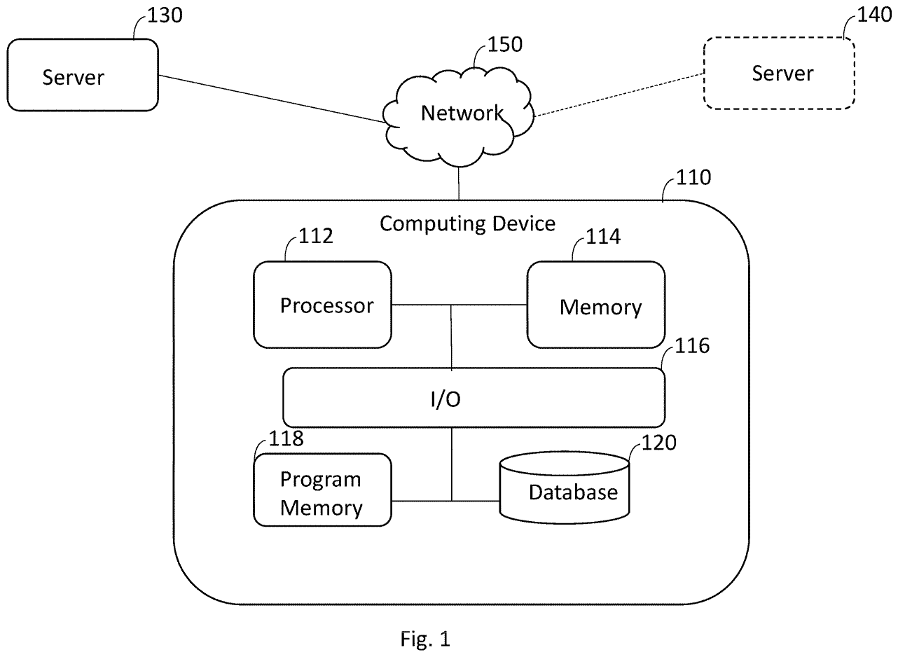

The type of system (e.g., computer system) is not particularly limited and may be any system comprising a computing device and one or more servers such as the system provided in FIG. 1. Referring to FIG. 1, a block diagram of a computer system 100 for generating a sampling rate for one or more of haploid induction, embryogenesis induction, haploid doubling, embryo survival, flowering, or harvest, or any combination thereof using at least one learned prediction model, and calculating the number of plants or male gametic cells that are needed to produce a desired number of haploid embryos or double haploid plants based upon the one or more sampling rates is shown. To do so, the system 100 may include a computing device 110 and a server 130 that is associated with a computer system. The system 100 may further include one or more servers 140 that are associated with other computer systems such that the computing device 110 may communicate with different computer systems running different platforms. However, it should be appreciated that, in some embodiments, a single server (e.g., a server 130) may run multiple platforms. The computing device 110 is communicatively coupled to the one or more servers 130, 140 via a network 150 (e.g., a local area network (LAN), a wide area network (WAN), a personal area network (PAN), the Internet, etc.).

In general, the computing device 110 may include any existing or future devices capable of training a machine learning model. For example, the computing device may be, but not limited to, a computer, a notebook, a laptop, a mobile device, a smartphone, a tablet, wearable, smart glasses, or any other suitable computing device that is capable of communicating with the server 130.

The computing device 110 includes a processor 112, a memory 114, an input/output (I/O) controller 116 (e.g., a network transceiver), a memory unit 118, and a database 120, all of which may be interconnected via one or more address/data bus. It should be appreciated that although only one processor 112 is shown, the computing device 110 may include multiple processors. Although the I/O controller 116 is shown as a single block, it should be appreciated that the I/O controller 116 may include a number of different types of I/O components (e.g., a display, a user interface (e.g., a display screen, a touchscreen, a keyboard), a speaker, and a microphone).

The processor 112 as disclosed herein may be any electronic device that is capable of processing data, for example a central processing unit (CPU), a graphics processing unit (GPU), a tensor processing unit (TPU), a system on a chip (SoC), or any other suitable type of processor. It should be appreciated that the various operations of example methods described herein (i.e., performed by the computing device 110) may be performed by one or more processors 112. The memory 114 may be a random-access memory (RAM), read-only memory (ROM), a flash memory, or any other suitable type of memory that enables storage of data such as instruction codes that the processor 112 needs to access in order to implement any method as disclosed herein. It should be appreciated that, in some embodiments, the computing device 110 may be a computing device or a plurality of computing devices with distributed processing.

As used herein, the term “database” may refer to a single database or other structured data storage, or to a collection of two or more different databases or structured data storage components. In the illustrative embodiment, the database 120 is part of the computing device 110. In some embodiments, the computing device 110 may access the database 120 via a network such as network 150. The database 120 may store data (e.g., input, output, intermediary data) used for calculating sampling rates. For example, the data may include genotypic data, such as single nucleotide polymorphisms (SNPs), genetic markers, haplotype, sequence information, phenotypic data, double haploid induction rates, predicted genetic values, pedigree information, co-ancestry information, or combinations thereof that are obtained from one or more servers 130, 140.

The computing device 110 may further include a number of software applications stored in a memory unit 118, which may be called a program memory. The various software applications on the computing device 110 may include specific programs, routines, or scripts for performing processing functions associated with the methods described herein. Additionally, or alternatively, the various software applications on the computing device 110 may include general-purpose software applications for data processing, database management, data analysis, network communication, web server operation, or other functions described herein or typically performed by a server. The various software applications may be executed on the same computer processor or on different computer processors. Additionally, or alternatively, the software applications may interact with various hardware modules that may be installed within or connected to the computing device 110. Such modules may implement part of or all of the various exemplary method functions discussed herein or other related embodiments.

Although only one computing device 110 is shown in FIG. 1, the server 130, 140 is capable of communicating with multiple computing devices similar to the computing device 110. Although not shown in FIG. 1, similar to the computing device 110, the server 130, 140 also includes a processor (e.g., a microprocessor, a microcontroller), a memory, and an input/output (I/O) controller (e.g., a network transceiver). The server 130, 140 may be a single server or a plurality of servers with distributed processing. The server 130, 140 may receive data from and/or transmit data to the computing device 110.

In certain embodiments, the computing device 110 may generate predictions of plant genotype performance by using at least one learned prediction model to generate a predicted haploid induction sampling for at least one or more candidate plant genotypes of interest. In certain embodiments, the computing device 110 may further generate a recommended number of plants of the candidate plant genotype needed to produce a desired number of haploid embryos.

In certain embodiments, the computing device 110 may generate predictions of plant genotype performance by using at least one learned prediction model to generate a predicted embryogenesis induction sampling for at least one or more candidate plant genotypes of interest. In certain embodiments, the computing device 110 may further generate a recommended number of male gametic cells of the candidate plant genotype needed to produce a desired number of haploid embryos.

In certain embodiments, the computing device 110 may generate predictions of plant genotype performance by using at least one learned prediction model to generate a predicted sampling rate for one or more of haploid induction, haploid doubling, embryo survival, flowering, or harvest, or any combination thereof for at least one or more candidate plant genotypes of interest. In certain embodiments, the computing device 110 may further generate a recommended number of plants of the candidate double haploid breeding population that are needed to produce a desired number of double haploid plants.

In certain embodiments, the computing device 110 may generate predictions of plant genotype performance by using at least one learned prediction model to generate a predicted sampling rate for one or more of embryogenesis induction, haploid doubling, embryo survival, flowering, or harvest, or any combination thereof for at least one or more candidate plant genotypes of interest. In certain embodiments, the computing device 110 may further generate a recommended number of male gametic cells of the candidate double haploid breeding population that are needed to produce a desired number of double haploid plants.

In certain embodiments, the computing device 110 may further generate an estimation of the uncertainty of the predicted sampling rate for one or more of haploid induction embryogenesis induction, haploid doubling, embryo survival, flowering, or harvest, or any combination thereof, and adjusting the one or more sampling rates based upon the estimation of uncertainty.

The following are examples of specific embodiments of some aspects of the invention. The examples are offered for illustrative purposes only and are not intended to limit the scope of the invention in any way.

### Example 1

This example demonstrates the development of a learned prediction model to generate predicted sampling rates.

Data collected at Corteva's DH production sites was used to develop and validate prediction models. The data was generated from a high throughput process and included germplasm with diverse genetic backgrounds. Before analysis, the data was checked for validity, completeness, and consistency of phenotypic and pedigree information. Part of the data was then used to develop models (estimation set) and the remaining was kept for validation (prediction set).

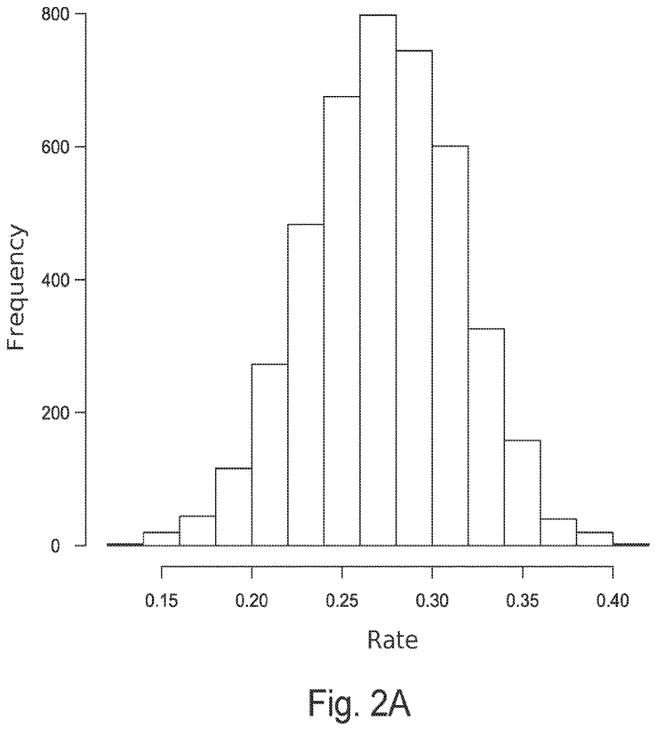

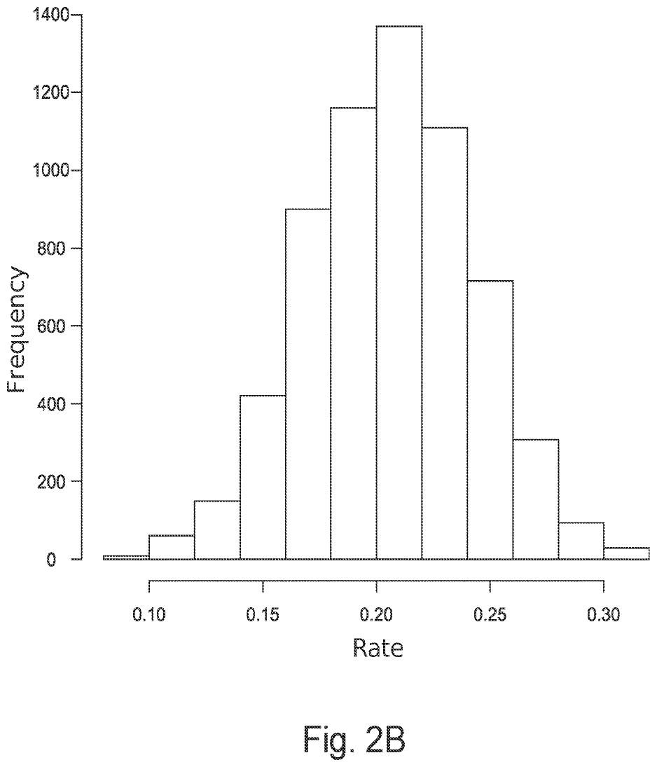

The data was analyzed separately by heterotic group and site. FIGS. 2A and 2B provide histograms of data used in the development of prediction models for the haploid induction rate trait. The data is summarized in Table 1. The data included thousands of observations with a mean haploid rate ranging from 20.6% to 27.3%.

TABLE 1
Summary of Data Used for Model Development

| | Number of | | |
|---|---|---|---|
| Heterotic Group | Samples (n) | Mean, % | Standard Deviation, % |
| 1 | 4371 | 27.3 | 4.2 |
| 2 | 6400 | 20.6 | 3.6 |

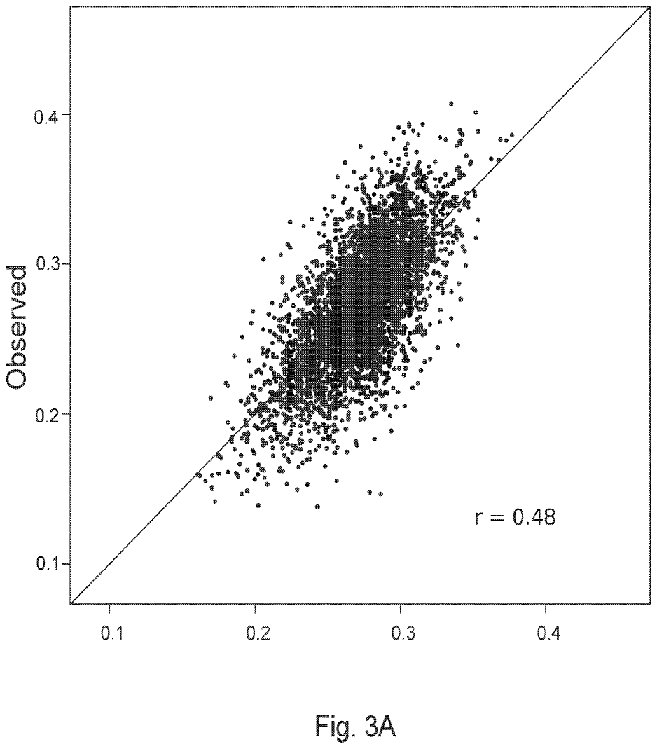

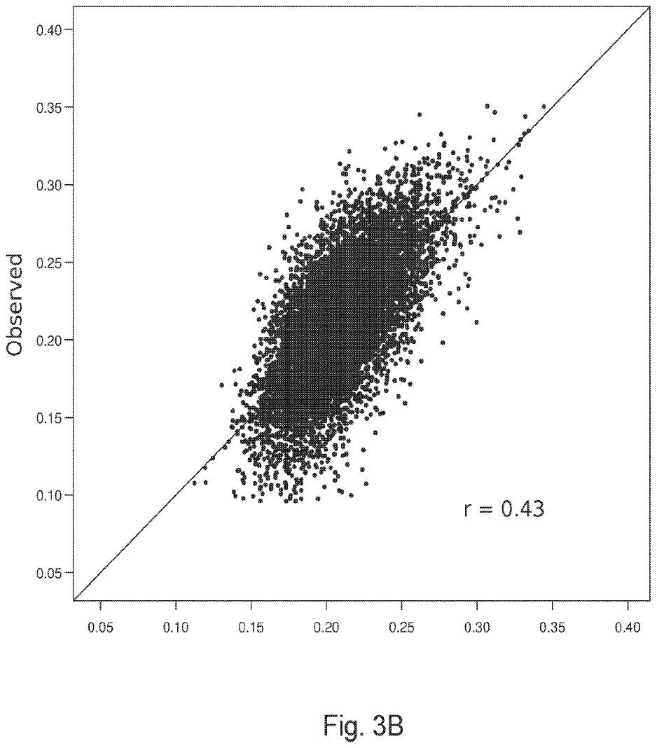

During the analysis, all DH traits were assumed to follow a binomial distribution with data consisting of counts of successes and failures in each step of the process. Data were analyzed based on Generalized Linear Models with a logit link. After the analysis, model fit was evaluated based on scatter plots and correlations between observed and predicted values. FIGS. 3A and 3B provide scatter plots between observed and predicted values with corresponding correlation coefficients for the heterotic groups.

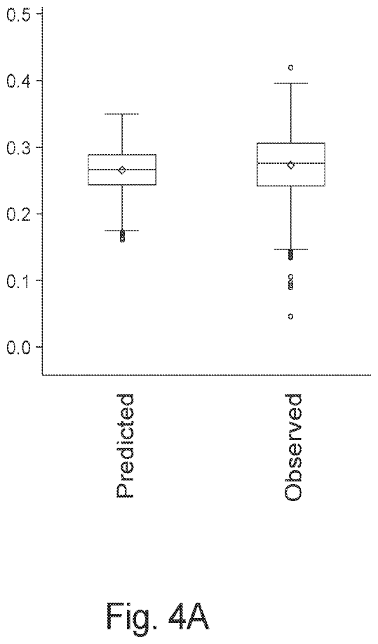

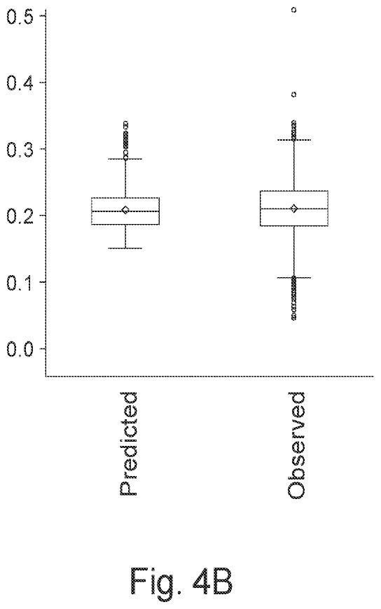

Models from the above step were then evaluated for prediction capability using the prediction set. Differences between observed and predicted values, correlation coefficients (r), and Standard Error of Prediction (SEP) were used to evaluate models. In addition, SEP values were averaged for use as a measure of uncertainty and to calculate confidence limits for new predictions. FIGS. 4A and 4B provide box plots for haploid rate models by heterotic group and the summary statistics are provided in Tables 2 and 3, below.

TABLE 2
Summary Statistics-Data Used for Model Validation

| Heter- | | | | | | | | | |
|---|---|---|---|---|---|---|---|---|---|
| otic | Observed | | | | | Predicted | | | |
| Group | n | Mean | Min | Max | SD | Mean | Min | Max | SD |
| 1 | 870 | 0.273 | 0.046 | 0.419 | 0.049 | 0.265 | 0.157 | 0.350 | 0.036 |
| 2 | 1078 | 0.211 | 0.046 | 0.509 | 0.044 | 0.209 | 0.151 | 0.338 | 0.031 |

TABLE 3
Correlations Between Observed and Predicted Values and
Standard Error of Predictions by Heterotic Group

| Heterotic Group | n | corr | SEP |
|---|---|---|---|
| 1 | 870 | 0.347 | 0.051 |
| 2 | 1078 | 0.238 | 0.047 |

The sampling rates come with a certain degree of uncertainty. To account for the uncertainty the predictions can be adjusted by computing the lower confidence limits at different probability levels. For example, let:
X be a random variable (haploid rate) distributed according to a distribution with cdf F.
x_{α} be haploid rate value such that we are (1−α)×100% confident the haploid rate is greater than this value:

$${x}_{α} = {F}_{α}^{- 1} , i . e . P ( X \leq {x}_{α} ) = α .$$

Now assume that X~N(μ,σ²).

Then

$$P ( X \leq {x}_{α} ) = P ( \frac{X - μ}{σ} \leq \frac{{x}_{α} - μ}{σ} ) = P ( Z \leq \frac{{x}_{α} - μ}{σ} ) = α ,$$

where Z~N(0,1), and
x_{α}=Φ⁻¹(α)σ+μ, where Φ⁻¹(x) is the cdf of the N(0,1).

Practically, μ is taken as the predicted value from the model and a as SEP

### Example 2

This example demonstrates using the learned prediction models to calculate the number of plants needed to produce a desired number of haploid embryos.

Table 4 provides a list of 20 germplasms from both heterotic groups with predicted haploid rate (\hat{p}) and final number of embryos to be rescued to meet target. Assume the target is to produce 500 haploid embryos at the end of this laboratory step. Assume an average SEP of 4.5%.
a. First run predictions for the new set of germplasms using the final model to produce predicted values. This is shown in column “predicted”. For instance, the predicted haploid rate for ID #1=(\hat{p})=25.7%,
b. Calculate the lower limit (one-sided) for the predicted values at different probability levels, e.g., (1−α)=0.95, 0.90, 0.85, and 0.80 were considered in the table. For ID #1 the corresponding lower limits were:

$$95 \% Lower limit ( α = \text{.05} ) = Lower 95 = \hat{p} + ( {z}_{α} \times SEP ) = 25.7 + ( - 1.64485 \times 4.5 ) = 18.298 \%$$

$$90 \% Lower limit ( α = \text{.10} ) = Lower 90 = \hat{p} + ( {z}_{α} \times SEP ) = 25.7 + ( - 1.28155 \times 4.5 ) = 19.933 \%$$

$$85 \% Lower limit ( α = \text{.15} ) = L o w e r 85 = \hat{p} + ( {z}_{α} \times SEP ) = 25.7 + ( - 103643 \times 4.5 ) = 21.036 \%$$

$$80 \% Lower limit ( α = \text{.20} ) = L o w e r 80 = \hat{p} + ( {z}_{α} \times S E P ) = 25.7 + ( - 0.84162 \times 4.5 ) = 21.913 \%$$

c. Calculate number of embryos to rescue at the corresponding probability levels.

$$Embryos to rescue = \frac{target value}{lower limit} \times 100$$

$$Embryos to rescue ( 95 \% prob ) = Rescue 95 = \frac{500}{18.298} \times 100 = 2 , 733$$

$$Embryos to rescue ( 90 \% prob ) = Rescue 90 = \frac{500}{19.933} \times 100 = 2 , 508$$

$$Embryos to rescue ( 85 \% prob ) = Rescue 85 = \frac{500}{21.036} \times 100 = 2 , 377$$

$$Embryos to rescue ( 80 \% prob ) = Rescue 80 = \frac{500}{21.913} \times 100 = 2 , 282$$

TABLE 4
Number of Embryos to Extract to Meet Desired Target

| | Heterotic | | | | | | | | | |
|---|---|---|---|---|---|---|---|---|---|---|
| ID | group | Predicted | Lower95 | Lower90 | Lower85 | Lower80 | Rescue95 | Rescue90 | Rescue85 | Rescue80 |
| 1 | 1 | 25.7 | 18.298 | 19.933 | 21.036 | 21.913 | 2733 | 2508 | 2377 | 2282 |
| 2 | 1 | 29.3 | 21.898 | 23.533 | 24.636 | 25.513 | 2283 | 2125 | 2030 | 1960 |
| 3 | 1 | 22.8 | 15.398 | 17.033 | 18.136 | 19.013 | 3247 | 2935 | 2757 | 2630 |
| 4 | 1 | 26.8 | 19.398 | 21.033 | 22.136 | 23.013 | 2578 | 2377 | 2259 | 2173 |
| 5 | 1 | 30.8 | 23.398 | 25.033 | 26.136 | 27.013 | 2137 | 1997 | 1913 | 1851 |
| 6 | 1 | 20.6 | 13.198 | 14.833 | 15.936 | 16.813 | 3788 | 3371 | 3138 | 2974 |
| 7 | 1 | 25.0 | 17.598 | 19.233 | 20.336 | 21.213 | 2841 | 2600 | 2459 | 2357 |
| 8 | 1 | 23.6 | 16.198 | 17.833 | 18.936 | 19.813 | 3087 | 2804 | 2640 | 2524 |
| 9 | 1 | 27.1 | 19.698 | 21.333 | 22.436 | 23.313 | 2538 | 2344 | 2229 | 2145 |
| 10 | 1 | 31.3 | 23.898 | 25.533 | 26.636 | 27.513 | 2092 | 1958 | 1877 | 1817 |
| 11 | 2 | 17.9 | 10.498 | 12.133 | 13.236 | 14.113 | 4763 | 4121 | 3778 | 3543 |
| 12 | 2 | 19.4 | 11.998 | 13.633 | 14.736 | 15.613 | 4167 | 3668 | 3393 | 3203 |
| 13 | 2 | 19.3 | 11.898 | 13.533 | 14.636 | 15.513 | 4202 | 3695 | 3416 | 3223 |
| 14 | 2 | 21.2 | 13.798 | 15.433 | 16.536 | 17.413 | 3624 | 3240 | 3024 | 2871 |
| 15 | 2 | 24.5 | 17.098 | 18.733 | 19.836 | 20.713 | 2924 | 2669 | 2521 | 2414 |
| 16 | 2 | 24.7 | 17.298 | 18.933 | 20.036 | 20.913 | 2890 | 2641 | 2495 | 2391 |
| 17 | 2 | 21.1 | 13.698 | 15.333 | 16.436 | 17.313 | 3650 | 3261 | 3042 | 2888 |
| 18 | 2 | 16.8 | 9.398 | 11.033 | 12.136 | 13.013 | 5320 | 4532 | 4120 | 3842 |
| 19 | 2 | 18.6 | 11.198 | 12.833 | 13.936 | 14.813 | 4465 | 3896 | 3588 | 3375 |
| 20 | 2 | 21.4 | 13.998 | 15.633 | 16.736 | 17.613 | 3572 | 3198 | 2988 | 2839 |

### Example 3

This example demonstrates calculating multiple steps in the DH production pathway.

Table 5 demonstrates example calculation for all 5 process steps using example ID #1 from Example 2. Values for the induction steps are as in Example 2. Assume that for the doubling step, the SEP is 1.8% and the predicted value is 47.7%, that for the survival step the SEP is 2.7% and the predicted value 57.9%, that for the flowering step the SEP is 4.2% and the predicted value 61.9% and that for the harvest step the SEP is 5.2% and the predicted value 39.7%. Assume further that the final target number of doubled haploids at the end of the process is 100 and that the 90% risk level is chosen. Then, 305 samples are needed for the harvest step, 540 for the flowering step, 993 for the survival step, 2,187 for the doubling step and 10,973 for the induction step. If, for example, the species is maize and one maize plant can produce 800 seeds, then a total of 10,973/800=14 plants (when rounded to next integer value) have to be pollinated with an inducer plant.

TABLE 5
Number of samples to collect in individual process steps to Meet Desired Target

| | | Process steps | | | | | |
|---|---|---|---|---|---|---|---|
| | | induction | doubling | survival | flowering | harvest | |
| SEP | (%) | 4.5 | 1.8 | 2.7 | 4.2 | 5.2 | |
| Predicted | (%) | 25.7 | 47.7 | 57.9 | 61.9 | 39.4 | final |
| Lower90 | (%) | 19.9 | 45.4 | 54.4 | 56.5 | 32.7 | target |

| | | | | | | |
|---|---|---|---|---|---|---|
| sampling | 10,973 | 2,187 | 993 | 540 | 305 | 100 |
| target | | | | | | |

### Example 4

This example demonstrates using the learned prediction models to calculate the number of microspores needed to produce a desired number of double haploid plants

Table 6 demonstrates example calculation for all 5 process steps. Assume that for embryogenesis step, the SEP is 4.7% and the predicted value is 23.1%, that for the doubling step, the SEP is 1.6% and the predicted value is 46.9%, that for the survival step the SEP is 3.1% and the predicted value 51.8%, that for the flowering step the SEP is 3.9% and the predicted value 63.7% and that for the harvest step the SEP is 5.5% and the predicted value 38.8%. Assume further that the final target number of doubled haploids at the end of the process is 100 and that the 90% risk level is chosen. Then, 315 samples are needed for the harvest step, 537 for the flowering step, 1,122 for the survival step, 2,501 for the doubling step and 14,647 microspores for the embryogenesis induction step.

TABLE 6
Number of samples to collect in individual process steps to Meet Desired Target

| | | Process steps | | | | | |
|---|---|---|---|---|---|---|---|
| | | embryogenesis | | | | | |
| | | induction | doubling | survival | flowering | harvest | |
| SEP | (%) | 4.7 | 1.6 | 3.1 | 3.9 | 5.5 | |
| Predicted | (%) | 23.1 | 46.9 | 51.8 | 63.7 | 38.8 | final |
| Lower90 | (%) | 17.1 | 44.8 | 47.8 | 58.7 | 31.8 | target |

| | | | | | | |
|---|---|---|---|---|---|---|
| sampling | 14,647 | 2,501 | 1,122 | 537 | 315 | 100 |
| target | | | | | | |

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A method for predicting haploid induction rate, the method comprising:
a. genotyping a double haploid breeding population comprising a plurality of plants from a parental cross;
b. inputting the genotypic information from the double haploid breeding population into a learned prediction model to generate a predicted haploid induction sampling rate for the double haploid breeding population;
c. calculating based on the predicted haploid induction sampling rate of the double haploid breeding population the number plants in the population that are needed to produce a desired number of haploid embryos;
d. pollinating the calculated number of plants of the double haploid breeding population with an inducer line; and
e. collecting the haploid embryos of the pollinated plants.

2. The method of claim 1, wherein the plurality of plants is from the same parental cross.

3. The method of claim 1, wherein the learned prediction model is trained using a training dataset comprising double haploid crosses with genotypic and phenotypic information.

4. The method of claim 1, wherein the learned prediction model comprises a machine learning model or a linear statistical model.

5-6. (canceled)

7. The method of claim 4, wherein the linear statistical model comprises a logistic generalized linear prediction model using the following formula:
$${s}_{i} = B ( {p}_{i} , {n}_{i} )$$
$${p}_{i} = ℒ ( {β}_{0} + {y}_{i} {β}_{y} + {\sum }_{j = 1}^{M} {z}_{ij} {u}_{j} )$$
wherein nᵢ is the number of embryos rescued from the iₜₕ, cross,
sᵢ the number of haploid embryos among the nᵢ,
pᵢ is the probability of observing a haploid embryo for cross i with
$$ℒ ( {β}_{0} + {y}_{i} {β}_{y} + {\sum }_{j = 1}^{M} {z}_{ij} {u}_{j} )$$
being its linear predictor and [symbol] (⋅) denoting the logistic link function,
β₀ is the general intercept,
β_{y} is optional and is an effect of an explanatory variable,
zᵢⱼ denotes the genotype score of marker j for cross i,
uⱼ is the corresponding marker effect, and
B(⋅) denotes the binomial probability function, with nᵢ known.

8. The method of claim 1, wherein the method further comprises calculating an estimation of the uncertainty of the predicted haploid induction sampling rate for the at least one double haploid breeding population and adjusting the predicted haploid induction sampling rate based on the estimated uncertainty.

9-10. (canceled)

11. The method of claim 1, wherein the genotype of the double haploid breeding population is imputed from the marker genotypes of the parents of the plants of double haploid breeding population.

12-27. (canceled)

28. A method for improved double haploid production efficiency, the method comprising:
a. genotyping a candidate double haploid breeding population comprising a plurality of plants from a parental cross;
b. inputting the genotypic information from the candidate double haploid breeding population into a first learned prediction model to generate a predicted haploid induction sampling rate;
c. inputting the genotypic information from the candidate double haploid breeding population into a second learned prediction model to generate a predicted sampling rate for at least one of, haploid doubling, embryo survival, flowering, or harvest;
d. calculating based on the predicted sampling rates of the double haploid breeding population the number plants in the population that are needed to produce a desired number of double haploid plants;
e. pollinating the calculated number of plants of the candidate double haploid breeding population with an inducer line; and
f. producing the desired number of double haploid plants.

29. The method of claim 28, wherein the second learned prediction model generates a predicted sampling rate for haploid doubling.

30. The method of claim 28, wherein after (a) and prior to (d) the method further comprises inputting the genotypic information from the candidate double haploid breeding population into a third learned prediction model to generate a predicted sampling rate for at least one of, embryo survival, flowering, or harvest.

31. (canceled)

32. The method of claim 30, wherein after (a) and prior to (d) the method further comprises inputting the genotypic information from the candidate double haploid breeding population into a fourth learned prediction model to generate a predicted sampling rate for at least one of, flowering, or harvest.

33-35. (canceled)

36. The method of claim 28, wherein the first and second learned prediction models are the same model and the predicted sampling rates are determined jointly using a learned prediction model comprising a Markov process model.

37-43. (canceled)

44. The method of claim 28, wherein the method further comprises calculating an estimation of the uncertainty of the predicted haploid induction rate for the at least one double haploid breeding population and adjusting the predicted haploid induction rate based on the estimated uncertainty.

45. (canceled)

46. The method of claim 28, wherein the method further comprises calculating an estimation of the uncertainty of the predicted sampling for at least one of haploid doubling, embryo survival, flowering, or harvest and adjusting the predicted sampling rate for at least one of haploid doubling, embryo survival, flowering, or harvest based on the estimated uncertainty.

47-48. (canceled)

49. The method of claim 28, wherein the genotype of the double haploid breeding population is imputed from the marker genotypes of the parents of the plants of double haploid breeding population.

50. A method for predicting embryogenesis induction rate, the method comprising:
a. genotyping a double haploid breeding population comprising a plurality of plants from a parental cross;
b. inputting the genotypic information from the double haploid breeding population into a learned prediction model to generate a predicted embryogenesis induction sampling rate for the double haploid breeding population;
c. calculating based on the predicted embryogenesis induction sampling rate of the double haploid breeding population the number male gametic cells that are needed to produce a desired number of haploid embryos;
d. inducing androgenesis in the calculated number of male gametic cells of the double haploid breeding population; and
e. collecting the haploid embryos.

51. The method of claim 50, wherein the learned prediction model is trained using a training dataset comprising double haploid crosses with genotypic and phenotypic information.

52. The method of claim 50, wherein the learned prediction model comprises a machine learning model or a liner statistical model.

53-54. (canceled)

55. The method of claim 52, wherein the linear statistical model comprises a logistic generalized linear prediction model using the following formula:
$${s}_{i} = B ( {p}_{i} , {n}_{i} )$$
$${p}_{i} = ℒ ( {β}_{0} + {y}_{i} {β}_{y} + {\sum }_{j = 1}^{M} {z}_{ij} {u}_{j} )$$
wherein nᵢ is the number of embryos rescued from the iₜₕ cross,
sᵢ the number of haploid embryos among the nᵢ,
pᵢ is the probability of observing a haploid embryo for cross i with
$$ℒ ( {β}_{0} + {y}_{i} {β}_{y} + {\sum }_{j = 1}^{M} {z}_{ij} {u}_{j} )$$
being its linear predictor and [symbol] (⋅) denoting the logistic link function,
β₀ is the general intercept,
β_{y} is optional and is an effect of an explanatory variable,
zᵢⱼ denotes the genotype score of marker j for cross i,
uⱼ is the corresponding marker effect, and
B(⋅) denotes the binomial probability function, with nᵢ known.

56. The method of claim 50, wherein the method further comprises calculating an estimation of uncertainty of the predicted embryogenesis induction sampling rate for the at least one double haploid breeding population and adjusting the predicted embryogenesis induction sampling rate based on the estimation of uncertainty.

57-80. (canceled)
